(19) United States
(12) Patent Application Publication
(10) Pub. No.: US 2006/0194853 A1
(43) Pub. Date: Aug. 31, 2006

(54) Alkyl benzamides

(76) Inventor: Jean Francois Rossignol, St. Petersburg, FL (US)

Correspondence Address: STEPHAN A. PENDORF, P.A., PENDORF & CUTLIFF, 5111 MEMORIAL HIGHWAY, TAMPA, FL, 33634, US

(21) Appl. No.: 11/244,787

(22) Filed: Oct. 6, 2005

Related U.S. Application Data
(60) Provisional application No. 60/617,412, filed on Oct. 8, 2004.

(51) Int. Cl. A61K 31/426; C07D 277/18

(52) U.S. Cl. 514/370; 548/191

(57) ABSTRACT

The present invention relates to alkyl derivatives of nitazoxanide and tizoxanide and isomers thereof. More specifically, the present invention is directed to compounds possessing anti-parasitic, antibacterial, antiviral and antifungal activities, and to methods of treatment and/or prevention of parasitic, bacterial, viral and/or fungal diseases in humans and animals using said compounds and pharmaceutical compositions thereof.

### REFERENCE TO RELATED APPLICATION

This application is a non-provisional of provisional application No. 60/617,412 filed Oct. 8, 2004.

### BACKGROUND OF THE INVENTION

1. Field of the Invention

The present invention relates to alkyl derivatives of nitazoxanide and tizoxanide and isomers thereof. More specifically, the present invention is directed to compounds possessing anti-parasitic, antibacterial, antiviral and antifungal activities, and to methods of treatment and/or prevention of parasitic, bacterial, viral and/or fungal diseases in humans and animals using said compounds and pharmaceutical compositions thereof.

2. Discussion of the Related Art

It is known that tizoxanide (2-hydroxy-N-(5-nitro-2-thiazolyl) benzamide, Compound A, U.S. Pat. No. 5,578,621) and nitazoxanide (2-acetyloxy-N-(5-nitro-2-thiazolyl)benzamide, Compound B, U.S. Pat. No. 3,950,351) possess potent activity against parasites, bacteria, viruses and fungi, as described, for example, in U.S. Pat. Nos. 4,315,018; 5,578,621; and 5,856,348.

Tizoxanide (Compound A) and nitazoxanide (Compound B) are compounds according to formula (I) in which: 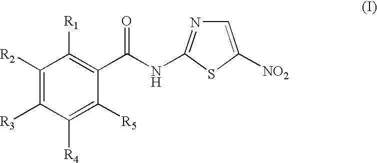
Compound A (tizoxanide): wherein R₁=—OH and R₂-R₅=—H; and
Compound B (nitazoxanide): wherein R₁=—OCOCH₃ and R₂-R₅=—H.

There is a need in the art for benzamide compounds that possess improved potency and/or improved target specificity compared to the compounds of the prior art. For example, for the treatment of parasitic or viral diseases, benzamide derivatives lacking antibacterial activity would be beneficial in order to avoid disruption of the gut flora when administered orally.

There is therefore a need in the art for benzamide compounds that possess superior activity against parasitic, bacterial, viral and fungal diseases suitable for treatment of humans and animals, and which do not suffer from the above-mentioned drawbacks of the compounds of the prior art. All this and more will be apparent to one of ordinary skill upon reading the following description, non-limiting examples, and claims.

### SUMMARY OF THE INVENTION

The present inventors have surprisingly discovered that certain alkyl derivatives of tizoxanide and nitazoxanide, and isomers of such derivatives, possess improved anti-parasitic, antibacterial, antiviral and antifungal activities.

Thus, in a first embodiment, the present invention is directed to compounds according to formula (I): 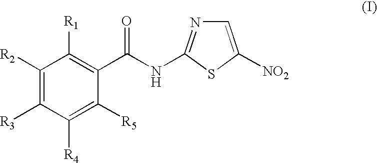
in which the R₁ substituent is —OH or —OCOCH₃, one of the R₂-R₅ substituents is alkyl and the remaining positions are —H, and salts, solvates or hydrates thereof.

In a second embodiment, the invention is directed to a method of treating or preventing a parasitic, bacterial, viral or fungal disease in a human or animal subject by administering an effective amount of the compound of formula (I) according to the first embodiment.

In a third embodiment, the invention is directed to pharmaceutical compositions comprising at least one compound according to formula (I) and a pharmaceutically acceptable carrier.

While the compounds of the first embodiment possess R₁ substituents that are —OH or —OCOCH₃, (as in Compound A (tizoxanide) and Compound B (nitazoxanide), respectively), the present invention is not so limited.

Thus, in a fourth embodiment, the invention is directed to compounds according to formula (I) comprising an —OH or —OCOCH₃ substituent at any one position among R₁-R₅, and in which one of the remaining R₁-R₅ substituents is alkyl and the remainder are —H, including salts, solvates and hydrates thereof.

In a fifth embodiment, the invention is directed to a method of treating or preventing a parasitic, bacterial, viral or fungal disease in a human or animal subject by administering to the subject an effective amount of the compound of according to the fourth embodiment.

In a sixth embodiment, the invention is directed to pharmaceutical compositions comprising a compound according to the fourth embodiment and a pharmaceutically acceptable carrier.

### DETAILED DESCRIPTION OF THE INVENTION

The present invention is directed to compounds according to formula (I), their methods of use, and pharmaceutical compositions thereof: 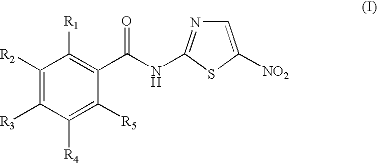
in which: one of R₁-R₅ is —OH or —OCOCH₃, and is preferably —OH; one of R₁-R₅ is alkyl, preferably C₁-C₄ alkyl, most preferably —CH₃; and
substituents R₁-R₅ that are not alkyl, —OH or —OCOCH₃ are —H.

The compounds of the present invention include organic and inorganic salts, solvates and hydrates of the compounds according to formula (I).

The term “alkyl” includes both branched alkyl and linear alkyl.

The compounds of the present invention possess improved activity against certain parasitic, bacterial, viral and fungal diseases, including but not limited to pathogens against which tizoxanide and/or zitaxoanide exhibit activity, as described, for example, in U.S. Pat. Nos. 4,315,018; 5,578,621; and 5,856,348.

Suitable formulations and dosages will be readily understood by one of ordinary skill from the formulations and dosages of prior art benzamide compounds as set forth in U.S. Pat. No. 6,117,894 (acid-stabilized compounds) and U.S. Pat. No. 5,968,961 (particle size).

Certain U.S. patents have been referred to herein, which are hereby incorporated for their cited teachings, and in their respective entireties, by reference.

The invention is now illustrated by the following, non-limiting, examples:

### EXAMPLE 1

### Compound D (2-acetyloxy-3-methyl-N-(5-nitro-2-thiazolyl) benzamide)

Compound D is a compound according to formula (I) in which R₁=—OCOCH₃, R₂=—CH₃ and R₃, R₄ and R₅=—H.

Compound D is synthesized from 3-methyl salicylic acid and 2-amino-5-nitro thiazole as shown in the following scheme: 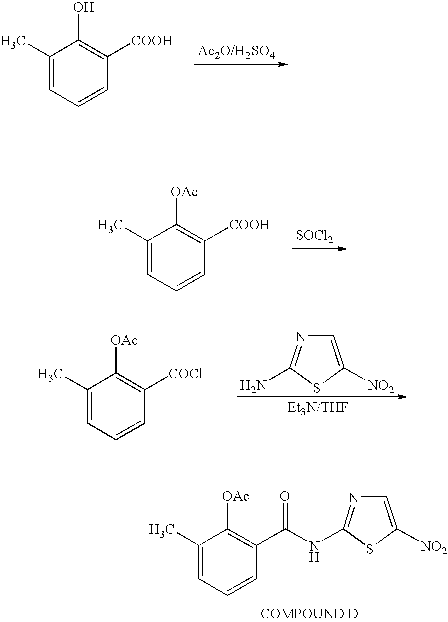
in which: Ac₂O is acetic anhydride, SOCl₂ is thionyl chloride, Et₃N is triethylamine, and THF is tetrahydrofuran. Other synthesis methods, reagents and adaptations will readily occur to those of skill in the art, and the compounds of the present invention are not limited by their method of synthesis, which is provided for illustrative purposes only. Throughout this disclosure —OAc and —OCOCH₃ are equivalent.

Compound D is effective against at least H. Pylori, C. jejuni, Influenza A and B, Herpes VZV, G. intestinalis, P. falciparum and T. vaginalis.

### EXAMPLE 2

### Compound C (2-hydroxy-3-methyl-N-(5-nitro-2-thiazolyl) benzamide)

Compound C is a compound according to formula (I) in which R₁=—OH, R₂=—CH₃ and R₃, R₄, R₅=—H.

Compound C can be synthesized by de-acetylating Compound D, for example in the presence of a strong acid such as concentrated hydrochloric acid.

Compound C possesses comparable or improved activity compared to nitazoxanide against Helicobacter pylori (IC₅₀=2 μg/mL), Campylobacter jejuni (IC₅₀=4 μg/mL), Influenza A (IC₅₀=0.18 μg/mL), Influenza B (IC₅₀=0.18 μg/mL), and Herpes Varicella A (IC₅₀=0.9 μg/mL).

### EXAMPLE 3

### Compound H (2-acetyloxy-5-methyl-N-(5-nitro-2-thiazolyl) benzamide)

Compound H is a compound according to formula (I) in which R₁=—OCOCH₃, R₄=—CH₃ and R₂, R₃, R₅=—H.

Compound H can be synthesized from 5-methyl salicylic acid and 2-amino-5-nitro thiazole as shown in the following scheme:

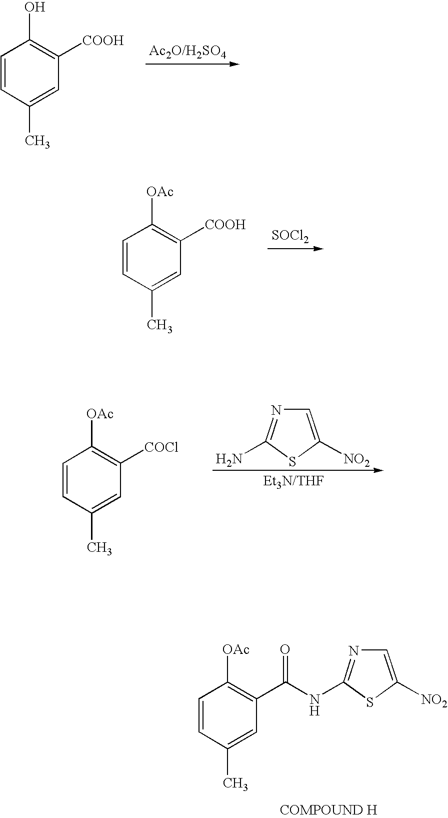

Compound H is effective against at least G. intestinalis and T. vaginalis.

### EXAMPLE 4

### Compound G (2-hydroxy-5-methyl-N-(5-nitro-2-thiazolyl) benzamide)

Compound G is a compound according to formula (I) in which R₁=—OH, R₄=—CH₃ and R₂, R₃, R₅=—H.

Compound G can be synthesized by de-acetylating Compound H for example in the presence of a strong acid such as concentrated hydrochloric acid.

Compound G is effective against at least G. intestinalis and T. vaginalis.

### EXAMPLE 5

### Compound F (2-acetyloxy-4-methyl-N-(5-nitro-2-thiazolyl) benzamide)

Compound F is a compound according to formula (I) in which R₁=—OCOCH₃, R₃=—CH₃ and R₂, R₄, R₅=—H.

Compound F can be synthesized from 4-methyl salicylic acid and 2-amino-5-nitro thiazole as shown in the following scheme:

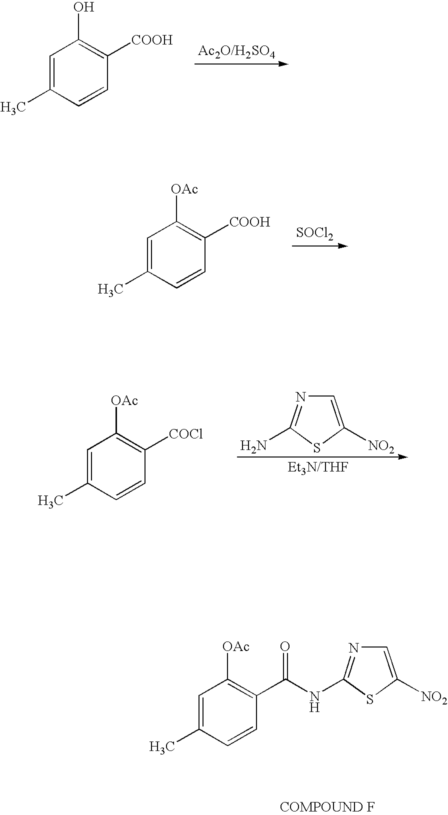

Compound F is effective against at least G. intestinalis and T. vaginalis.

### EXAMPLE 6

### Compound E (2-hydroxy-4-methyl-N-(5-nitro-2-thiazolyl) benzamide)

Compound E is a compound according to formula (I) in which R₁=—OH, R₃=—CH₃ and R₂, R₄, R₅=—H.

Compound E can be synthesized by de-acetylating Compound F for example in the presence of a strong acid such as concentrated hydrochloric acid.

Compound E is effective against at least G. intestinalis and T. vaginalis.

### EXAMPLE 7

### Comparative Testing

Compounds C, D, E and G were tested in vitro against the protozoa, Giardia intestinalis and Trichomonas vaginalis with the results shown in TABLE I.

TABLE I
IC₅₀ (μM)*

| | Giardia intestinalis | Trichomonas |
|---|---|---|
| Compound | JKH-1 | vaginalis UCH-1 |
| C | 0.31 | 6.148 |
| D | 0.56 | 8.04 |
| E | 0.195 | 0.287 |
| G | 0.20 | 0.751 |
| Tizoxanide (Compound A) | Not done | 1.521 |
| Nitazoxanide | 12.46 | 0.605 |
| (Compound B) | | |

*Micromolar concentrations of drugs required to inhibit 50% of the growth of the organisms.

Compounds C, D, E and G are significantly more potent than nitazoxanide against Giardia intestinalis.

Compounds E and G exhibit improved activity, compared to tizoxanide, against Trichomonas vaginalis, with Compound E (methyl group at R₄) being the most effective. The methyl group at R₂, R₃ or R₄ therefore improves the activity of nitazoxanide and tizoxanide. A comparison of the results for Compounds C and D shows that these compounds are most potent when R₁=OH instead of —OCOCH₃.

What is claimed is:

1. A compound according to Formula (I): 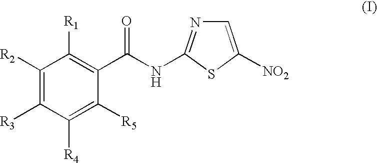
wherein
R₁ is —OH or —OCOCH₃; and
R₂-R₅ are —H except that one of R₂-R₅ is alkyl, or a salt, solvate or hydrate thereof.

2. The compound according to claim 1, wherein said alkyl is C₁-C₄ alkyl.

3. The compound according to claim 2, wherein R₅ is —H.

4. The compound according to claim 3, wherein said alkyl is —CH₃.

5. The compound according to claim 4, wherein R₁ is —OH.

6. The compound according to claim 5, wherein R₂ is —CH₃.

7. The compound according to claim 5, wherein R₃ is —CH₃.

8. The compound according to claim 5, wherein R₄ is —CH₃.

9. The compound according to claim 4, wherein R₁ is —OCOCH₃.

10. The compound according to claim 9, wherein R₂ is —CH₃.

11. The compound according to claim 9, wherein R₃ is —CH₃.

12. The compound according to claim 9, wherein R₄ is —CH₃.

13. A method of treating or preventing a parasitic, bacterial, viral or fungal disease in a human or animal subject comprising administering to said subject an effective amount of a compound according to Formula (I): 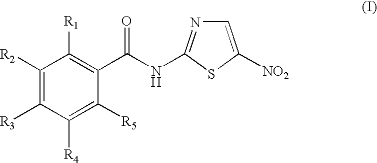
wherein
R₁ is —OH or —OCOCH₃; and
R₂-R₅ are —H except that one of R₂-R₅ is alkyl, or a salt, solvate or hydrate thereof.

14. The method of claim 13, wherein said disease is a disease that is susceptible to treatment by tizoxanide or nitazoxanide.

15. The method of claim 14, wherein said disease is associated with a pathogen selected from the group consisting of Giardia intestinalis, Trichomonas vaginalis, Helicobacter pylori, Campylobacter jejuni, Influenza B A, and Herpes Varicella A.

16. A pharmaceutical composition comprising at least one compound according to claim 1 and a pharmaceutically acceptable carrier.

17. A compound according to Formula (I): 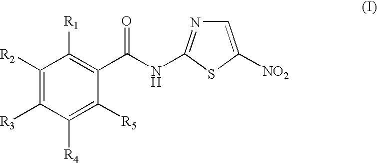
wherein
R₁-R₅ are —H, except that one of R₁-R₅ is —OH or —OCOCH₃ and one of R₁-R₅ is alkyl, or a salt, solvate or hydrate thereof.

18. The compound according to claim 17, wherein said alkyl is C₁-C₄ alkyl.

19. The compound according to claim 18, wherein said alkyl is —CH₃.

20. A method of treating a parasitic, bacterial, viral or fungal disease in human or animal subject comprising administering to said subject an effective amount of the compound according to claim 17.

21. A pharmaceutical composition comprising at least one compound according to claim 17 and a pharmaceutically acceptable carrier.
